(19) United States
(12) Patent Application Publication
(10) Pub. No.: US 2004/0024028 A1
(43) Pub. Date: Feb. 5, 2004

(54) Method for reducing or eliminating smoking

(76) Inventor: Steven L. Burton, Pittsburgh, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/333,443

(22) PCT Filed: Aug. 2, 2001
(86) PCT No.: PCT/US01/24360

(51) Int. Cl. A61K 31/4439

(52) U.S. Cl. 514/343

(57) ABSTRACT

This invention is an improved method for reducing or eliminating tobacco usage and the nicotine dependency associated with tobacco usage by gradually decreasing an individual's tobacco consumption over a time period while replacing nicotine from tobacco with an alternative nicotine source in the form of a transdermal patch.

### BACKGROUND OF THE INVENTION

The present invention relates to a method for gradually reducing or eliminating an individual's tobacco usage habit, in particular smoking, as well as the associated nicotine dependence that is created by tobacco use, utilizing an alternative nicotine source in the form of a transdermal patch. The idea of applying reduction strategies to smoking cessation has been explored in the development of smoking cessation methods over the last twenty years. However, reduction as an end goal or as a means to cessation has received very little attention in recent years. In fact, these methods are not even included as treatment options in the Agency for Health Care Policy and Research (AHCPR) Centers for Disease Control and Prevention Smoking Cessation Guideline, the authoritative review that outlines the standard of care for smoking cessation. See U.S. Department of Health and Human Services, AHCPR Publication No. 96-0692, April 1996.

Innovative treatments of smoking reduction or cessation are significantly lacking. Most smoking control methods are cessation methods and are undertaken without the aid of pharmacological or behavioral therapy, in part because the quitting approaches associated with these therapies are not consistent with a smoker's preferred quitting approach. Recent research on smoker's attitude toward quitting shows that in comparison to abrupt cessation, many smokers who are interested in quitting smoking prefer to quit by gradually reducing the amount they smoke. Recent studies demonstrate that gradual reduction strategies can succeed without pharmacological therapy. These strategies, however, involve intensive behavioral intervention and always have as their goal smoking cessation rather than reduction only. For example, see Cinciripini et al., “The Effects of Smoking schedules on Cessation Outcome: Can We improve on Common Methods of Gradual And Abrupt Nicotine Withdrawal,” Journal of Consulting and Clinical Psychology, Vol. 63, No. 3, pp. 388-399 (1995), comparing two gradual reduction quitting methods to quitting “cold turkey.” The gradual reduction methods of quitting described in Cinciripini et al. included (1) progressively increasing inter-cigarette intervals and (2) gradually reducing the number of cigarettes smoked without changing the inter-cigarette interval. All methods included the use of cognitive-behavioral relapse prevention training, and were without pharmacological therapy such as an alternative nicotine source.

These efforts to quit by gradual reduction without nicotine replacement often fail. It has been suggested that one reason that gradual reduction fails is because tobacco users are progressively deprived of nicotine and consequently experience nicotine cravings and withdrawal which deter quitting or further reduction.

One method that combines reduction with the use of nicotine replacement therapy is described in U.S. Pat. No. 5,055,478. In one embodiment, the method described in this patent includes the steps of first recording over an approximately two week period, the times during which each cigarette is consumed. These time periods of smoking are referred to as consumption periods, which are not necessarily consecutive in time. Then, tobacco consumption gradually is decreased by replacing in an increasing number of consumption periods (up to 16 consumption periods) over a two week period, the use of tobacco with an alternative nicotine source such as Nicorette® nicotine-containing chewing gum (2 mg nicotine dosage), until no further tobacco is consumed. The nicotine in each piece of gum is a substitute for all the tobacco consumed during a consumption period. This level of consumption of the alternative nicotine source is maintained for approximately four weeks. During this four week period, while the individual is still receiving nicotine through the alternative nicotine source, the individual addresses the social and psychological reasons for smoking. After the four week period, the alternative nicotine source is eliminated at a rate of one consumption period per week for approximately 14-16 weeks, until no further alternative nicotine source is consumed.

A second method described in U.S. Pat. No. 5,055,478 includes an initial step of recording for approximately two weeks an individual's normal tobacco consumption pattern to identify the times of day during which tobacco is consumed, and the amount of tobacco consumed during these periods. Each day is then broken down into consumption periods of one hour each. Then, all tobacco consumption is abruptly stopped and an alternative nicotine source (approximately 1.5 to 1.75 mg. per consumption period) is administered for a period of about two weeks. As with the first method above, following cessation of smoking but while the individual is still receiving nicotine through the alternative nicotine source, the individual addresses the social and psychological reasons for smoking. The user then gradually decreases administration of the alternative nicotine source down to a lower level of approximately 1 mg per consumption period for approximately 10-18 days. This is done by administering the lower dosage alternative nicotine source during an increasing number of consumption periods until the lower dosage is consumed during all consumption periods. After reaching the lower dosage in each consumption period, the lower dosage alternative nicotine source is administered for about two weeks. Then, the user gradually eliminates consumption of the alternative nicotine source by eliminating consumption of the source during an increasing number of consumption periods (one per week) a until no alternative nicotine source is consumed.

Another method closely related to the methods described in U.S. Pat. No. 5,055,478, combines smoking reduction with the use of nicotine replacement therapy. This method is described in Cooper et al., “New Hope for Heavy Smokers The Cooper/Clayton Method to Stop Smoking”. In this method, after the initial two week recordation phase, an alternative nicotine source is substituted for all consumption periods, and the individual immediately stops smoking. The remainder of the method is similar to the above method but elimination of the alternative nicotine source is to a maintenance level which is about one-third of the first maintenance level, followed by gradual reduction as in the final phase described above. In this method, the individual receives intensive person-to-person psychological intervention.

Yet another method of smoking cessation is described in a publication by SmithKline Beecham entitled “Nicorette® Committed Quitters™ Calendar and Smoking-Cessation Service. In this method, the user abruptly stops smoking and consumes one piece of Nicorette® gum (2 mg. or 4 mg. dosage, generic name nicotine polacrilex gum) every 1-2 hours consuming no more than 24 pieces per day for a six week period. In week seven the individual cuts back to consuming one piece of Nicorette® gum every two hours and continues at this level for three weeks. In week 10, the individual cuts back to one piece of Nicorette® gum every four hours. After week 12, the individual stops using Nicorette® gum. During the twelve week period, the individual records the number of pieces of Nicorette® gum chewed and some activities the individual plans to do each day to help cope with not smoking. The calendar also provides helpful hints for dealing with cravings and the psychological aspects of quitting smoking, and a reminder of the amount of time invested in quitting.

### SUMMARY OF THE INVENTION

This invention is an improved method for reducing or eliminating tobacco usage, particularly smoking, and the nicotine dependency associated with tobacco usage including a step of gradually decreasing an individual's tobacco usage over a time period while replacing nicotine from tobacco with an alternative nicotine source in the form of a transdermal patch.

### DETAILED DESCRIPTION OF THE INVENTION

The present invention as described herein, is a method to reduce or eliminate an individual's tobacco usage habit, in particular smoking, as well as the nicotine dependency associated with that habit. The method includes the step of gradually decreasing tobacco usage over a time period until the individual significantly reduces or eliminates tobacco usage. The reduction or elimination of tobacco usage is accomplished with or without behavioral intervention using nicotine replacement therapy such as a nicotine-containing transdermal patch in conjunction with gradual tobacco use reduction. Such a strategy expands the use of nicotine replacement therapy from an abrupt tobacco usage cessation to a gradual reduction. Gradually reducing the number of cigarettes smoked or other nicotine products used while replacing nicotine from tobacco with an alternative nicotine source such as a nicotine-containing transdermal patch aids in reducing nicotine cravings and withdrawal thereby facilitating reduction or elimination of tobacco usage.

The methods of this invention are intended to be applicable to reduction and/or cessation of the use of tobacco products including smokeable tobacco such as cigarettes and cigars, and smokeless tobacco such as chewing tobacco and snuff.

The alternative nicotine source is in the form of a transdermal patch comprising a nicotine substance. The alternative nicotine source will transdermally deliver a dosage of nicotine substance to aid in reducing cravings and withdrawal from tobacco usage.

As used herein, a “nicotine substance” may be nicotine, a substance equivalent to or approximating nicotine including any and all known compounds and/or compositions, such as lobeline sulfate, that produce a similar physiological effect as nicotine, or a mixture thereof that produces a similar physiological effect as nicotine. Preferably the nicotine substance is nicotine.

Transdermal patches such as are known in the art may be readily adapted by known methods for use in the present invention. Transdermal patches suitable for use in the present invention include those described in U.S. Pat. Nos. 5,004,610; 5,342,623; 5,344,656; 5,364,630; 5,462,745; and 5,508,038. Preferred transdermal patches comprise from about 7 mg to about 21 mg nicotine dosage. The transdermal patches comprise about 7, about 14 or about 21 mg nicotine dosage for use in preferred methods of the invention. Preferred transdermal patches are those used in the nicotine transdermal system marketed by SmithKline Beecham, Pittsburgh, Pa., under the tradename NICODERM CQ. Such patches comprise ethylene vinyl-acetate-copolymer, polyisobutylene and high density polyethylene between pigmented and clear polyester backings. Other suitable patches are described in U.S. Pat. Nos. 5,077,104; 5,268,209; 4,908,027; 5,633,008; 4,839,174; 4,943,435; and 5,167,242. The transdermal patches will in general be used in accordance with typical procedures, for example being applied to dry, clean and hairless skin; worn for about 24 hours (if there are disruptions of sleep while wearing the patch 24 hours, the patch may be removed at bedtime (after about 16 hours), and a new one put on after rising the next day); and removing and replacing the patch at approximately the same time every day.

The methods of the invention include a step of gradually decreasing the individual's tobacco usage over a time period while replacing all or part of the nicotine from tobacco with an alternative nicotine source in the form of a transdermal patch. The levels and pattern of tobacco usage reduction, the time period over which reduction occurs, and the regimen for administering the alternative nicotine source may be adjusted, e.g., to reflect differences in the tobacco usage pattern of the individual and/or the individual's preferences regarding reduction. The individual being treated preferably addresses the social and psychological reasons for using tobacco during treatment.

In one embodiment, the method of the invention comprises the step of:

(a) administering an alternative nicotine source in the form of a transdermal patch comprising a nicotine substance once a day for a first predetermined period of time, wherein the first predetermined period of time the individual significantly reduces tobacco usage (which can be described as a “reduction phase”); and optionally

(b) administering a second alternative nicotine source in the form of a transdermal patch comprising a nicotine substance once a day for a second predetermined period of time, wherein the second alternative nicotine source preferably comprises a higher nicotine substance dosage than the first alternative nicotine source; and wherein the second predetermined period of time the individual uses no tobacco (which can be described as a “quit phase” or “cessation phase”).

In the methods of the invention, the length of the predetermined periods of time, nicotine substance dosages, and reduction in tobacco usage prior to quitting may be adjusted, e.g., in order to reflect differences in the tobacco usage pattern of the individual and/or the individual's preference regarding time to reduce or quit.

The first predetermined period of time (the reduction phase) is preferably substantially about 2 to 3 weeks in length. During this period, the individual preferably reduces tobacco usage by about 33% or more (i.e., as used herein about ⅓ or more), more preferably about 50% or more (i.e., as used herein about ½ or more), or also about 67% or more (i.e., as used herein about ⅔ or more)(said percentages or reductions relative to average tobacco consumption at the beginning of the tobacco reduction or cessation treatment). The transdermal patches used during this period are selected to aid in reducing cravings and withdrawal from tobacco usage. In preferred embodiments the transdermal patch used in the reduction phase comprises a nicotine dosage of about 7 or about 14 mg, more preferably about 14 mg.

In preferred embodiments, the reduction phase comprises two or more predetermined periods of time wherein the nicotine substance dosage in the alternative nicotine source and/or the reduction in tobacco usage is varied between the predetermined periods of time. For example, where the nicotine substance is nicotine, the reduction phase may comprise:

(a) two predetermined periods of time wherein a first such period tobacco usage is reduced by about 50% and the transdermal patch comprises about 14 mg nicotine dosage, and wherein the second such period the new level of tobacco usage is maintained or further reduced and the transdermal patch comprises about 14 mg nicotine dosage; or

(b) three predetermined periods of time wherein a first such period tobacco usage is reduced by about 33% and the transdermal patch comprises about 7 mg nicotine dosage, wherein the second such period tobacco usage is reduced by about 67% and the transdermal patch comprises about 14 mg nicotine dosage, and wherein the third such period tobacco usage is maintained at the new level or further reduced and the transdermal patch comprises about 14 mg nicotine dosage; or

(c) three predetermined periods of time wherein a first such period tobacco usage is reduced by about 33% and the transderrnal patch comprises about 14 mg nicotine dosage, wherein the second such period tobacco usage is reduced by about 67% and the transdermal patch comprises about 14 mg nicotine dosage, and wherein such third period tobacco usage is maintained at the new level or further reduced and the transdermal patch comprises about 14 mg nicotine dosage.

In the cessation phase, the individual ceases tobacco use, supported by nicotine replacement therapy in the form of a transdermal patch comprising nicotine substance. The nicotine replacement therapy is preferably selected to gradually assist the individual in becoming nicotine-free. The cessation phase will typically comprise a predetermined period of time wherein the individual is administered a transdermal patch having a relatively high dosage of nicotine substance, e.g., about 21 mg nicotine, preferably followed by at least one predetermined period of time wherein the individual is administered a transdermal patch having a lower dosage of nicotine substance, e.g. about 7 or about 14 mg nicotine. The number and length of a predetermined period of time and nicotine substance dosage may be adjusted in order to reflect differences in the individual's needs or preferences for becoming tobacco- and nicotine-free. The cessation phase will typically be substantially 8-12 weeks in length, more typically substantially about 10 weeks in length. Typically, a relatively high dosage of nicotine substance is administered for a predetermined period substantially about 6 weeks in length, followed by administration of one or more lower dosages of nicotine substance for a total predetermined period substantially about 2 to 4 weeks in length.

Where the nicotine substance is nicotine, in a preferred cessation phase the individual will use a 21 mg nicotine dosage transdermal patch for a predetermined period of substantially about 6 weeks, followed by 14 mg nicotine dosage transdermal patch for a predetermined period of substantially about 2 weeks in length, followed by a 7 mg nicotine dosage transdermal patch for a predetermined period of time substantially about 2 weeks in length.

The individual being treated preferably addresses the social and psychological reasons for using tobacco during one or more phases, preferably each of the phases, of the tobacco reduction or cessation treatment.

In a preferred embodiment, the method of the present invention comprises the steps of:

(a) administering a first alternative nicotine source in the form of a transdermal patch comprising a nicotine substance once a day for a first predetermined period of time, wherein the first predetermined period of time the individual significantly reduces tobacco usage;

(b) administering the first alternative nicotine source once a day for a second predetermined period of time, wherein the second predetermined period of time the individual maintains the level of tobacco usage achieved in step(a) or further reduces tobacco usage;

(c) administering a second alternative nicotine source in the form of a transdermal patch comprising a nicotine substance once a day for a third predetermined period of time, wherein the second alternative nicotine source comprises a higher dosage of nicotine substance than the first alternative nicotine source;

(d) administering a third alternative nicotine source in the form of a transdermal patch comprising a nicotine substance once a day for a fourth predetermined period of time, wherein the third alternative nicotine source comprises substantially the same dosage of nicotine substance as the first alternative nicotine source; and

(e) administering a fourth alternative nicotine source in the form of a transdermal patch comprising a nicotine substance once a day for a fifth predetermined period of time, wherein the fourth alternative nicotine source comprises a lower dosage of nicotine substance than the first alternative nicotine source; and

wherein during the third, fourth and fifth predetermined periods of time the individual uses no tobacco.

In a preferred embodiment the nicotine substance in each of the steps (a) (e) is nicotine. More preferably, the first transdermal patch comprises about 14 mg nicotine dosage, the second transdermal patch comprises about 21 mg nicotine dosage, the third transdermal patch comprises about 14 mg nicotine dosage, and the fourth transdermal patch comprises about 7 mg nicotine dosage.

Preferably the first predetermined period of time is substantially 1 week in length; the second predetermined period of time is substantially 1 week in length; the third predetermined period of time is substantially 6 weeks in length; the fourth predetermined period of time is substantially 2 weeks in length; and the fifth predetermined period of time is substantially 2 weeks in length. The individual being treated preferably addresses the social and psychological reasons for using tobacco during the first to the fifth predetermined periods of time.

During the first predetermined period of time, the individual preferably reduces tobacco use by about 50% or more, relative to their average tobacco use at the start of the tobacco reduction or cessation treatment. During the second predetermined period of time, the individual preferably maintains the new level of tobacco use or further reduces tobacco use by about 67% or more, relative to tobacco use at the start of the treatment.

In one embodiment of the present invention, the alternative nicotine source in the form of a transdermal patch is administered to a smoker who is instructed substantially as follows:

(a) (week 1) On days 1 through 7, use a 14 mg patch and start to cut back the number of cigarettes you smoke. By day 7, achieve a 50% reduction in the amount you smoke.

(b) (week 2) On days 8 through 14, continue to wear a 14 mg patch. Continue to reduce smoking in week 2 and prepare to quit.

(c) On day 15, stop smoking all cigarettes. Switch to a 21 mg patch. Stop smoking regardless of whether you reached your goals for reduced smoking. For best results, apply a 21 mg patch on the morning of your quit date. Continue to wear a 21 mg patch for 6 weeks to reduce you cravings and remain smoke free.

(d) (week 9) On week 9, switch to a 14 mg patch to reduce your nicotine dose. This stage is a lower strength patch that will continue to reduce your cravings. Wear a 14 mg patch for 2 weeks.

(e) (week 11) On week 11, switch to a 7 mg patch to reduce your nicotine intake even further. Wear a 7 mg patch for 2 weeks to become nicotine free.

During the 12 week period, while the individual is still receiving nicotine through the alternative nicotine source, the individual addresses the social and psychological reasons for smoking.

Also during the 12 week period, the individual preferably uses tools to assist in tracking and encouraging progress in smoking reduction and cessation. For example, at the start of week 1 the individual identifies the week 1 reduction goal and begins to use a daily calendar to track progress. The calendar preferably identifies the planned tobacco reduction for each day over weeks 1 and 2; tracks the level of tobacco use until the quit date; tracks each day the patch is worn to reduce cravings; tracks cravings in terms of number and intensity (e.g., on scale of 1-5), as the patch is used; and/or tracks the strength of the patch used. The calendar preferably identifies the phase of the treatment program, e.g,, reduce (week 1), continue to reduce (week 2), quit (week 3), remain tobacco free (weeks 4-10), become nicotine free (weeks 11-12). The calendar may also provide helpful hints for dealing with cravings and the psychological aspects of quitting tobacco, and/or a reminder of the amount of time invested in quitting. Stickers for use in a personal calendar, which identify the phase (by concept or otherwise) and patch strength, are preferably used to mark key dates. The products and tools, e.g., calendar and stickers, are preferably color coded with the phase of the treatment program.

Other tools which may be used include cards to provide to friends, family and the like to help generate their support as the individual seeks to reduce or cease tobacco use; lists identifying reasons for quitting; tips for remaining tobacco free; tips for ways to reduce tobacco use; and/or a container such as a box to use for storing tobacco that is planned to be used during the reduction period weeks 1 and 2.

The present invention is also directed to a method of reducing tobacco usage without necessarily eliminating it. In this embodiment, once an individual reaches the level of tobacco usage desired, the individual continues to use the desired amount of tobacco while continuing to use an alternative nicotine source in the form of a transdermal patch at that particular level. For example, if the individual reduces smoking to smoking four cigarettes per day and uses a 7 mg or 14 mg patch once a day, the individual can continue that regimen. Of course, this alternative embodiment does not preclude having reduction of tobacco usage as the ultimate goal and then immediately continuing the methods described in the above embodiments to cessation of tobacco usage, or maintaining the reduction regimen for a period and then continuing the method, according to the above embodiments, to cessation of tobacco usage.

The methods described above are especially suited for modern smoking habits brought on by low tolerance for smoking as is found, for example, in a smoke-free workplace. The changing social views of smoking has forced smokers to consume tobacco less frequently and often in inhospitable environments. The steadily increasing use of an alternative nicotine source used in the above methods dulls the cravings that naturally arise during smokeless periods. Reduced cravings allow the user to stay within the plan more easily than if tobacco is eliminated entirely or if an alternative nicotine source is used only during time-spaced consumption periods.

In the implementation of the methods according to the present invention, individuals need not be provided with intensive instruction but may be given only simple written instructions to follow setting forth the above steps. The present methods thus tend to overcome barriers in previous methods by providing efficacious nicotine replacement therapy for use in a gradual method of reduction or cessation of tobacco usage without intensive, face-to-face contact with a counselor or psychologist.

The foregoing description of present invention is presented for purposes of illustration and description. It is not intended to be exhaustive or to limit the present invention to the precise embodiments or steps disclosed. Obvious modifications or variations are possible in light of the above teachings. The above embodiments were chosen and described to provide the best illustration of the principles of the present invention and its practical application to thereby enable one of ordinary skill in the art to utilize the invention in various embodiments and with various modifications as are suited to the particular use contemplated. All such modifications and variations are within the scope of the present invention as determined by the appended claims when interpreted in accordance with the breadth to which they are fairly, legally and equitably entitled.

What is claimed is:

1. A method of gradually reducing or eliminating an individual's tobacco usage habit, comprising the step of gradually decreasing the individual's tobacco usage over a time period while replacing nicotine from tobacco with an alternative nicotine source in the form of a transdermal patch.

2. A method of gradually reducing or eliminating an individual's tobacco usage habit, comprising the step of:
(a) administering an alternative nicotine source in the form of a transdermal patch comprising a nicotine substance once a day for a predetermined period of time, wherein the predetermined period of time the individual continues to use tobacco but significantly reduces tobacco usage.

3. A method according to claim 2 further comprising a step of:
(b) administering a second alternative nicotine source in the form of a transdermal patch comprising a nicotine substance once a day for a second predetermined period of time, wherein the second predetermined period of time the individual uses no tobacco.

4. A method of claim 3 wherein second alternative nicotine source comprises a higher nicotine substance dosage than the first alternative nicotine source.

5. The method of any of claims 2-4 wherein the first predetermined period of time is substantially 2 to 3 weeks in length.

6. The method of any of claims 2-5 wherein the first predetermined period of time the individual reduces tobacco usage by about 33% or more.

7. The method of any of claims 2-5 wherein the first predetermined period of time the individual reduces tobacco usage by about 50% or more.

8. The method of any of claims 2-5 wherein the first predetermined period of time the individual reduces tobacco usage by about 67% or more.

9. The method of claim 2 or 3 wherein step (a) comprises two or more predetermined periods of time wherein the nicotine substance dosage in the alternative nicotine source and/or the reduction in tobacco usage is varied between the predetermined periods of time.

10. The method of claim 9 wherein step (a) comprises
(a) two predetermined periods of time wherein a first such period tobacco usage is reduced by about 50% and the transdermal patch comprises about 14 mg nicotine dosage, and wherein the second such period the new level of tobacco usage is maintained or further reduced and the transdermal patch comprises about 14 mg nicotine dosage; or
(b) three predetermined periods of time wherein a first such period tobacco usage is reduced by about 33% and the transdermal patch comprises about 7 mg nicotine dosage, wherein the second such period tobacco usage is reduced by about 67% and the transdermal patch comprises about 14 mg nicotine dosage, and wherein the third such period tobacco usage is maintained at the new level or further reduced and the transdermal patch comprises about 14 mg nicotine dosage; or
(c) three predetermined periods of time wherein a first such period tobacco usage is reduced by about 33% and the transdermal patch comprises about 14 mg nicotine dosage, wherein the second such period tobacco usage is reduced by about 67% and the transdermal patch comprises about 14 mg nicotine dosage, and wherein such third period tobacco usage is maintained at the new level or further reduced and the transdermal patch comprises about 14 mg nicotine dosage.

11. A method of any of the preceding claims wherein the nicotine substance is nicotine.

12. A method of gradually reducing or eliminating an individual's tobacco usage habit, comprising the steps of:
(a) administering a first alternative nicotine source in the form of a transdermal patch comprising a nicotine substance once a day for a first predetermined period of time, wherein the first predetermined period of time the individual significantly reduces tobacco usage;
(b) administering the first alternative nicotine source once a day for a second predetermined period of time, wherein the second predetermined period of time the individual further reduces tobacco usage;
(c) administering a second alternative nicotine source in the form of a transdermal patch comprising a nicotine substance once a day for a third predetermined period of time, wherein the second alternative nicotine source comprises a higher nicotine substance dosage than the first alternative nicotine source;
(d) administering a third alternative nicotine source in the form of a transdermal patch comprising a nicotine substance once a day for a fourth predetermined period of time, wherein the third alternative nicotine source comprises substantially the same nicotine substance dosage as the first alternative nicotine source; and
(e) administering a fourth alternative nicotine source in the form of a transdermal patch comprising a nicotine substance once a day for a fifth predetermined period of time, wherein the fourth alternative nicotine source comprises a lower nicotine substance dosage than the first alternative nicotine source;
wherein during the third, fourth and fifth predetermined periods of time the individual uses no tobacco.

13. A method according to claim 12 wherein the nicotine substance in steps (a) (e) is nicotine.

14. A method according to claim 13 wherein:
the first transdermal patch comprises about 14 mg nicotine dosage, the second transdermal patch comprises about 21 mg nicotine dosage, the third transdermal patch comprises about 14 mg nicotine dosage, and the fourth transdermal patch comprises about 7 mg nicotine dosage.

15. A method according to any of claims 11-14 wherein:
the first predetermined period of time is substantially 1 week in length;
the second predetermined period of time is substantially 1 week in length;
the third predetermined period of time is substantially 6 weeks in length;
the fourth predetermined period of time is substantially 2 weeks in length; and
the fifth predetermined period of time is substantially 2 weeks in length.

16. A method according to any of claims 11-15 wherein during the first to the fifth predetermined periods of time the individual addresses the social and psychological reasons for smoking.

17. A method according to any of claims 11-16 wherein during the first predetermined period of time, the individual reduces tobacco use by about 33% or more.

18. A method according to any of claims 11-16 wherein during the first predetermined period of time, the individual reduces tobacco use by about 50% or more.

19. A method according to any of the claims 11-16 wherein during the second predetermined period of time, the individual reduces tobacco use by about 66% or more.

20. A method according to any of the preceding claims in which the tobacco usage is smoking.

21. A method according to any of the preceding claims in which the tobacco usage is the use of a form of smokeless tobacco.
